(19) United States
(12) Patent Application Publication
(10) Pub. No.: US 2008/0306489 A1
(43) Pub. Date: Dec. 11, 2008

(54) Inserter for a spinal implant

(76) Inventors: Moti Altarac, Irvine, CA (US); Stanley Kyle Hayes, Mission Viejo, CA (US); Tyler Jay Haskins, Oceanside, CA (US); Robert C. Gutierrez, Huntington Beach, CA (US)

Correspondence Address: RIMAS LUKAS;VERTIFLEX, INC., 1351 CALLE AVANZADO, SAN CLEMENTE, CA, 92673, US

(21) Appl. No.: 12/215,497

(22) Filed: Jun. 27, 2008

Related U.S. Application Data
(63) Continuation-in-part of application No. 12/157,647, filed on Jun. 11, 2008.
Application No. 12/157,647 is a continuation-in-part of application No. 12/156,857, filed on Jun. 4, 2008.
(60) Provisional application No. 60/937,384, filed on Jun. 27, 2007.
(60) Provisional application No. 60/934,104, filed on Jun. 11, 2007.
(60) Provisional application No. 60/933,538, filed on Jun. 7, 2007.

(51) Int. Cl. A61B 17/58; A61B 17/00

(52) U.S. Cl. 606/99; 606/207

(57) ABSTRACT

An inserter for implanting a spinal implant such as an intervertebral spacer into a spinal disc space is disclosed. The inserter comprises a jaw assembly connected to a shaft assembly that is connected to a handle assembly. The user operates the handle assembly to open and close the jaw assembly to thereby connect to and release from the implant. Furthermore, the handle assembly is operable to lock and unlock rotation of the jaw assembly while still connected thereto to permit angulation of the jaw assembly relative to the shaft assembly without losing hold of the implant.

### CROSS-REFERENCE TO RELATED APPLICATIONS

This application claims priority to and the benefit of U.S. Provisional Patent Application Ser. No. 60/937,384 entitled “Inserter for intervertebral spacer” filed on Jun. 27, 2007 which is incorporated herein by reference in its entirety. This application also claims priority to and is a continuation-in-part of U.S. patent application Ser. No. 12/157,647 entitled “Inserter for a spinal implant” filed on Jun. 11, 2008 which claims priority to and the benefit of U.S. Provisional Patent Application Ser. No. 60/934,104 filed on Jun. 11, 2007. This application also claims priority to and is a continuation-in-part of U.S. patent application Ser. No. 12/156,857 entitled “Inserter for a spinal implant” filed on Jun. 4, 2008 which claims priority to and the benefit of U.S. Provisional Patent Application Ser. No. 60/933,538 filed on Jun. 7, 2007, all of which are hereby incorporated by reference in their entireties.

### FIELD

The present invention generally relates to medical devices, and in particular, the present invention relates to a surgical instrument for introducing spinal implants such as an intervertebral spacer into a disc space between adjacent vertebral bodies.

### BACKGROUND

Deterioration or dislocation of a spinal disc located between two adjacent vertebral bodies often results in the two adjacent vertebral bodies coming closer together. The reduced disc space height typically results in instability of the spine, decreased mobility and pain and discomfort for the patient. A common treatment is to surgically restore the proper disc space height to thereby alleviate the neurologic impact of the collapsed disc space. Typically, most surgical corrections of a disc space include at least a partial discectomy which is followed by restoration of normal disc space height and, in some instances, fusion of the adjacent vertebral bodies. Restoration of normal disc space height generally involves the implantation of a spacer and fusion typically involves inclusion of bone graft or bone graft substitute material into the intervertebral disc space to create bony fusion. Fusion rods may also be employed. Some implants further provide artificial dynamics to the spine. Such techniques for achieving interbody fusion or for providing artificial disc functions are well-known in the art.

One problem, among others, with inserting an implant, for example, is associated with patient anatomy. Inserting and positioning the implant in the space between adjacent vertebrae can be difficult or time consuming if the bony portions are spaced too close together, or if the adjacent tissue, nerves or vasculature impedes access to or placement of the implant in the space between the bony portions. Furthermore, maintenance of distraction of the space during insertion of the implant requires additional instruments in the operative space which can make the procedure more invasive and impede access and visibility during implant insertion and thereby make the procedure more difficult.

Another difficulty of implant insertion is related to the point of access to the damaged disc space which may be accomplished from several approaches to the spine with each approach having different associated difficulties. One approach is to gain access to the anterior portion of the spine through a patient's abdomen. For an anterior approach, extensive vessel retraction is often required and many vertebral levels are not readily accessible from this approach. Another approach is a posterior approach. This approach typically requires that both sides of the disc space on either side of the spinal cord be surgically exposed, which may require a substantial incision or multiple access locations, as well as extensive retraction of the spinal cord. Yet another approach is a postero-lateral approach to the disc space. The posterior-lateral approach is employed in a posterior lumbar interbody fusion (PLIF) or a transforaminal lumber interbody fusion (TLIF) procedure which may be performed as an open technique which requires making a larger incision along the middle of the back. Through this incision, the surgeon then cuts away, or retracts, spinal muscles and tissue to access the vertebrae and disc space. The TLIF procedure may also be performed as a minimally invasive or as an extreme lateral interbody fusion (XLIF) procedure that involves a retroperitoneal transpoas approach to the lumbar spine as an alternative to “open” fusion surgery. In the minimally invasive procedure, the surgeon employs much smaller incisions, avoids disrupting major muscles and tissues in the back and reduces the amount of muscle and tissue that is cut or retracted. As a result, blood loss is dramatically reduced and these minimally invasive benefits also lead to shorter hospital stays and quicker patient recovery times. The aforementioned and various other difficulties associated with the point of access to the damaged disc space and the need to navigate an implant insertion instrument through the point of access further place demands on the implant insertion instrument design.

Therefore, there remains a need for improved insertion instruments, implants and techniques for use in any one or more types of approaches to the disc space that facilitate and provide for effective insertion while saving time, minimizing the degree of invasiveness for the patient and complementing surgeon skill demands.

### SUMMARY

According to on aspect of the invention, an inserter for implanting a spinal implant is provided. The inserter includes a jaw assembly configured to attach to the spinal implant. The inserter also includes a shaft assembly connected to the jaw assembly. The shaft assembly includes a shaft connected to a gear wheel. The inserter further includes a handle assembly connected to the gear wheel and the shaft assembly such that the handle assembly is operable to open and close the jaw assembly to thereby connect to and release the spinal implant and operable to lock and unlock rotation of the gear wheel to permit angulation of the jaw assembly relative to the shaft assembly. The gear wheel includes a wedge-shaped shaft receiving portion with the shaft disposed therein; the wedge-shaped shaft receiving portion limiting the degree of rotation of the gear wheel with respect to the shaft and thereby limiting the degree of angulation of an attached spinal implant with respect to the shaft assembly. The gear wheel is rotatable with respect to the handle assembly to angulate a spinal implant attached to the jaw assembly with respect to the shaft. A trigger is provided and configured to engage the gear wheel to lock the angulation in place and configured to release the gear wheel to permit angulation. In one variation, the trigger is configured such that it is movable distally by the user to release the gear wheel to permit angulation. In one variation, a slap hammer surface is provided at the proximal end of the inserter. The shaft includes a right shaft and left shaft. The proximal end of the right shaft is connected to the gear wheel at a distance offset from the center of the gear wheel. The proximal end of the left shaft is connected to the gear wheel at a distance offset from the center of the gear wheel. In one variation, the shaft assembly includes an angled portion such that the distal working end of the inserter is displaced from the proximal handle end by a distance such that the distal working end of the inserter is substantially unobstructed by the inserter's proximal end.

According to another aspect of the invention, an inserter for implanting a spinal implant is disclosed. The inserter includes a jaw assembly configured to attach to a spinal implant. The inserter includes a shaft assembly connected to the jaw assembly. The shaft assembly includes an inner shaft comprising a right inner shaft and a left inner shaft, an outer shaft, and a gear wheel. The inner shaft has a distal end configured to connect with the jaw assembly. The outer shaft has a distal end configured to connect with the jaw assembly. The inner shaft is located in the outer shaft such that the inner shaft is movable with respect to the outer shaft. The proximal end of the right inner shaft is connected to the gear wheel. The proximal end of the left inner shaft is connected to the gear wheel. The inserter includes a handle assembly. The handle assembly includes a first portion connected to the gear wheel such that the gear wheel is rotatable with respect to the first portion. The handle assembly further includes a second portion connected to the first portion. The second portion is connected to the proximal end of the outer shaft such that the outer shaft is movable with respect to the second portion. The handle assembly includes a trigger connected to the first portion such that it is movable with respect to the first portion to unlock rotation of the gear wheel to permit angulation of the jaw assembly and lock rotation of the gear wheel to arrest angulation of the jaw assembly.

According to another aspect of the invention, a method for implanting a spinal implant is provided. The method includes the step of providing an instrument. The instrument includes a jaw configured to attach to a spinal implant, a shaft connected to the jaw, and a handle connected to the shaft. The jaw is operable via the handle to open and close to connect to the spinal implant and also operable via the handle to angulate an attached spinal implant with respect to the shaft and further operable to lock and unlock such angulation. A spinal implant configured to attach to the instrument and sized for frictional engagement between spinal anatomy is provided. The jaw of the instrument is opened. The spinal impant is placed in juxtaposition to the jaw. The jaw is closed and the spinal implant is connected to the instrument. An incision in a patient targeting spinal anatomy is created and the instrument with the attached spinal implant is inserted into the incision. The spinal implant is positioned between spinal anatomy such that the spinal anatomy exerts a frictional force on the spinal implant holding it substantially in place. The instrument is unlocked such that angulation of the implant relative to the shaft is permitted. The attached implant is angulated relative to the shaft while the implant is connected to the instrument. The instrument is locked such that angulation of the implant relative to the shaft is arrested. The spinal implant is re-positioned between the spinal anatomy. The jaw is opened. The instrument is removed from the incision and spinal implant is left located in position between the spinal anatomy.

### BRIEF DESCRIPTION OF THE DRAWINGS

The invention is best understood from the following detailed description when read in conjunction with the accompanying drawings. It is emphasized that, according to common practice, the various features of the drawings are not to-scale. Some dimensions of the various features are arbitrarily expanded or reduced for clarity.

FIG. 1a illustrates a perspective view of an inserter according to the present invention.

FIG. 1b illustrates a top view of the inserter of FIG. 1 according to the present invention.

FIG. 1c illustrates a side view of the inserter of FIG. 1 according to the present invention.

FIG. 1d illustrates a cross-sectional view of the inserter of FIG. 1 according to the present invention.

FIG. 2a illustrates a perspective view of a right jaw of the jaw assembly of the inserter according to the present invention.

FIG. 2b illustrates a perspective view of a left jaw of the jaw assembly of the inserter according to the present invention.

FIG. 3 illustrates a perspective view of an outer shaft of the shaft assembly of the inserter according to the present invention.

FIG. 4 illustrates a top view of an inner shaft assembly connected to a jaw assembly of the inserter according to the present invention.

FIG. 4a illustrates top and side views of a right inner shaft of the inner shaft assembly of the inserter according to the present invention.

FIG. 4b illustrates top and side views of a left inner shaft of the inner shaft assembly of the inserter according to the present invention.

FIG. 4c illustrates a side and cross-sectional view taken along line A-A of a gear wheel of the inner shaft assembly of the inserter according to the present invention.

FIG. 5 illustrates a perspective view of the handle of the handle assembly of the inserter according to the present invention.

FIG. 6 illustrates a cross-sectional view of a trigger of the handle assembly of the inserter according to the present invention.

FIG. 7 illustrates a perspective view of a spring of the handle assembly of the inserter according to the present invention.

FIG. 8 illustrates a knob of the handle assembly of the inserter according to the present invention.

FIG. 9a illustrates a top and detail view of a spacer in juxtaposition with the inserter according to the present invention.

FIG. 9b illustrates a top and detail view of a spacer connected to the inserter according to the present invention.

FIG. 9c illustrates a top and detail view of a spacer connected to the inserter and angulated in one direction according to the present invention.

### DETAILED DESCRIPTION

Before the subject devices, systems and methods are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs.

It must be noted that as used herein and in the appended claims, the singular forms “a”, “an”, and “the” include plural referents unless the context clearly dictates otherwise. Thus, for example, reference to “a spinal segment” may include a plurality of such spinal segments and reference to “the screw” includes reference to one or more screws and equivalents thereof known to those skilled in the art, and so forth.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The present invention is described in the accompanying figures and text as understood by a person having ordinary skill in the field of spinal implants and related instrumentation.

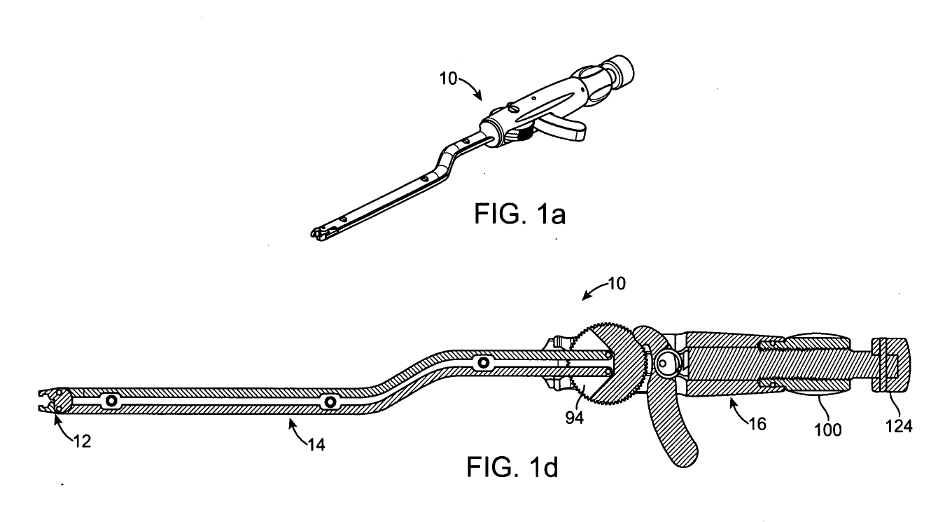

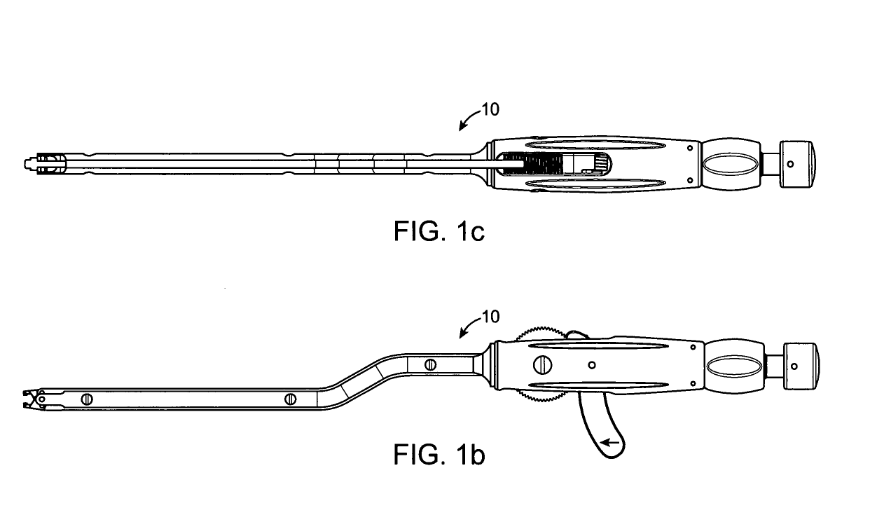

Referring now to FIGS. 1a-1d, there is shown a perspective view, a top view, a side view, and a cross-sectional view, respectively, of an inserter 10 for inserting a spinal implant into a patient such as an intervertebral spacer into a disc space between two adjacent vertebral bodies. With particular reference to FIG. 1d, the inserter 10 includes a jaw assembly 12, a shaft assembly 14 and a handle assembly 16.

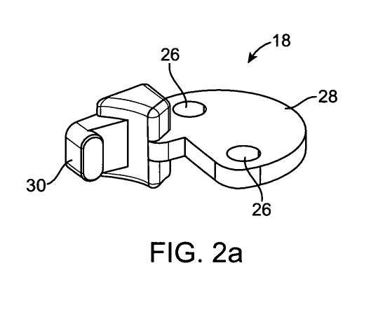

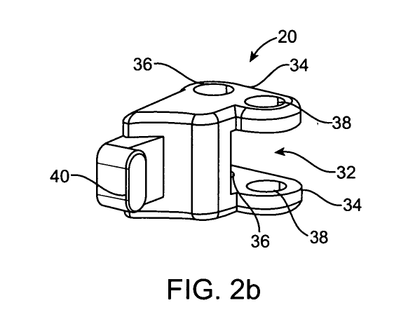

The jaw assembly 12 will now be discussed in reference to FIGS. 2a and 2b. The jaw assembly 12 includes a jaw comprising two jaw pieces-a right jaw piece 18 and a left jaw piece 20-two jaw pins 22 (not shown), and two shaft pins 24 (not shown).

With particular reference to FIG. 2a, the right jaw piece 18 includes a substantially flat portion with a curvate proximal end 28 connected to spacer engaging features 30. Two shaft pin apertures 26 are formed in the substantially flat portion 28. The spacer engaging features 30 are extending features configured to engage the interbody spacer (not shown). The features 30 are projections configured to be inserted into complementary shaped apertures in the spinal implant.

With particular reference to FIG. 2b, the left jaw piece 20 includes a right jaw receiving portion 32 formed between two substantially flat portions 34-an upper flat portion and a lower flat portion. Each flat portion 34 includes a shaft pin aperture 36 and a jaw pin aperture 38. The shaft pin aperture 36 of the upper flat portion is aligned with the shaft pin aperture 36 of the lower flat portion and the jaw pin aperture 38 of the upper flat portion is aligned with the jaw pin aperture 38 of the lower flat portion. The upper and lower flat portions 34 are connected to spacer engaging features 40. The spacer engaging features 40 are extending features configured to engage the interbody spacer (not shown). The features are projections configured to be inserted into complementary shaped apertures in the spinal implant.

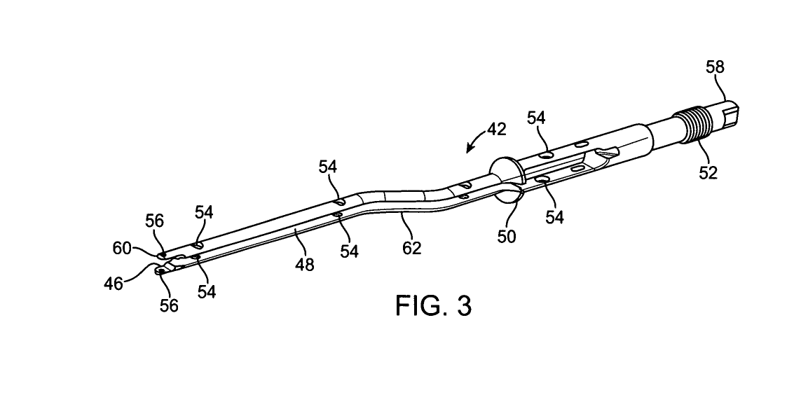

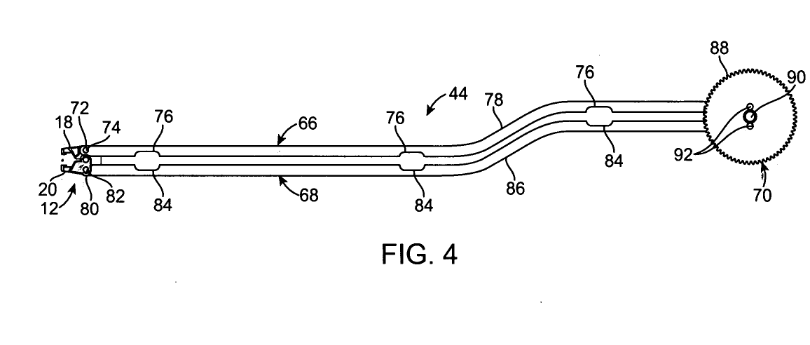

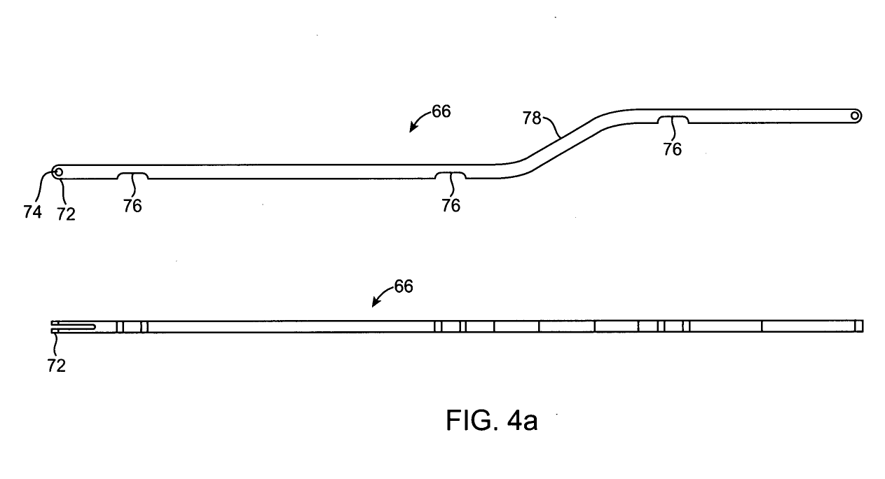

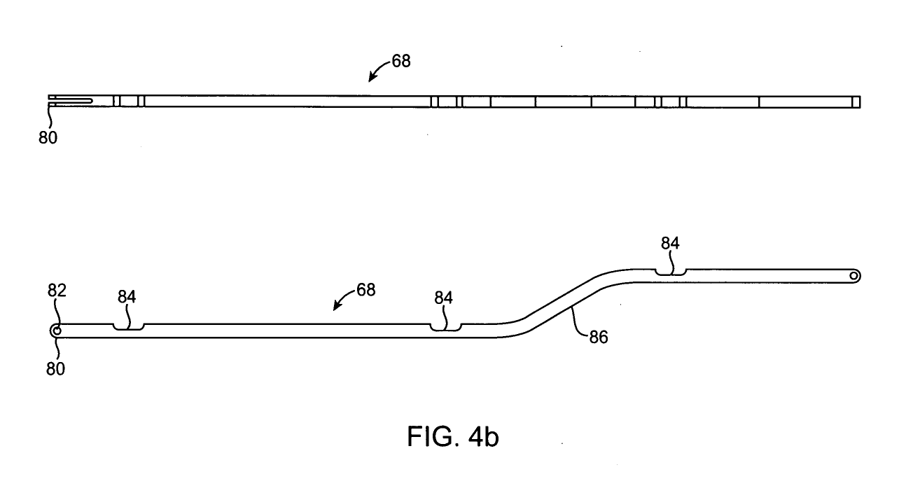

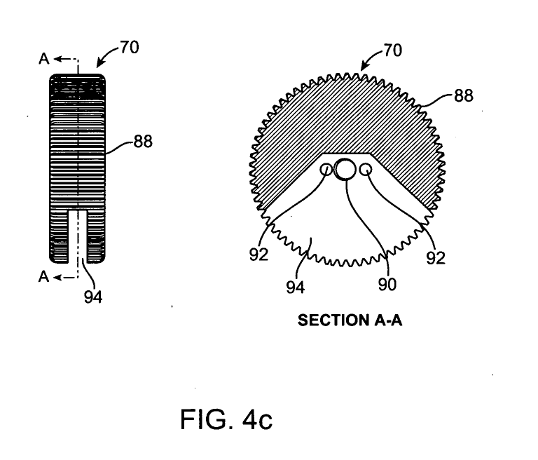

Turning to FIGS. 3 and 4, the shaft assembly 14 according to the present invention will now be discussed. The shaft assembly includes an outer shaft 42 shown in FIG. 3 and an inner shaft assembly 44 shown in FIG. 4. The outer shaft 42 is connected to the inner shaft 44.

With particular reference to FIG. 3, there is shown a perspective view of the outer shaft 42. The outer shaft 42 has a solid proximal end 58 and splits into two prongs towards the distal end 60. The split portion of the outer shaft 42 forms a jaw assembly receiving portion 46 and an inner shaft assembly receiving portion 48 between the prongs. A shoulder 50 for abutment against the handle assembly 16 is also formed. The proximal end 58 includes a threaded portion 52. The outer shaft 42 includes a plurality of connecting apertures 54 aligned in the upper and lower prongs for connected to the inner shaft assembly 44. The distal end 60 includes jaw pin apertures 56 aligned in the upper and lower prongs for receiving jaw pins 22 (not shown) for connecting the outer shaft 42 to the jaw assembly 12. In one variation, the outer shaft 42 includes an angled portion 62 imparting the outer shaft 42 with a bayonet-like appearance. The bayonet shape permits the working distal end 60 to be displaced from the proximal handling end 58. The displacement of the working distal end 60 from the proximal handling end 58 by a distance eases installation of the implant and helps keep the working distal end 60 substantially unobstructed by the instrument's proximal end when viewed from the proximal end 58 along the longitudinal axis of the distal end 60. In another variation, the outer shaft 42 is not angled and is a substantially straight tube.

With particular reference to FIG. 4, there is shown a top view of the inner shaft assembly 44 connected to the jaw assembly 12. The inner shaft assembly 44 includes a right inner shaft 66 and a left inner shaft 68 both connected to a gear wheel 70 at the proximal end.

With particular reference to FIG. 4a, the right inner shaft 66 includes a pronged distal end 72 that includes aligned upper and lower shaft pin apertures 74 for receiving shaft pins 24 and connecting to the right jaw piece 18. The body of the right inner shaft 66 includes elongated notches 76 substantially aligned with the connecting apertures 54 of the outer shaft 42. The proximal end of the right inner shaft 66 includes a gear pin aperture for receiving a gear pin and connecting to the gear wheel 70. In one variation, the right inner shaft 66 includes an angled portion 78 that conforms substantially to the angled portion 62 of the outer shaft 42.

With particular reference to FIG. 4b, the left inner shaft 68 includes a pronged distal end 80 that includes aligned upper and lower shaft pin apertures 82 for receiving shaft pins 24 and connecting to the left jaw piece 20. The body of the left inner shaft 68 includes elongated notches 84 substantially aligned with the connecting apertures 54 of the outer shaft 42 and elongated notches 76 of the right inner shaft 66. The proximal end of the left inner shaft 68 includes a gear pin aperture for receiving a gear pin and connecting to the gear wheel 70. In one variation, the left inner shaft 68 includes an angled portion 86 that conforms substantially to the angled portion 62 of the outer shaft 42 and the angled portion 78 of the right inner shaft 66.

With particular reference to FIG. 4c, the gear wheel 70 of the inner shaft assembly is substantially circular in shape and includes a toothed periphery 88. The gear wheel 70 includes a central bore 90 and gear pin apertures 92 for receiving gear pins and connecting to the right and left inner shafts 66, 68. The gear wheel 70 includes an inner shaft assembly receiving portion 94 (also seen in FIG. 1d) for receiving the proximal end of the inner shaft assembly 44 inside the gear wheel 70. The inner shaft assembly receiving portion 94 is substantially wedge-shaped for limiting rotation of the wheel 70 about the inner shaft assembly 44 within this wedge as well as defining the span of angulation of the jaw assembly 12.

The assembly of the inner shaft assembly 44 will now be described in reference to FIG. 4. The proximal ends of the right inner shaft 66 and left inner shaft 68 are connected to the gear wheel 70 by passing gear pins (not shown) into gear pin apertures of the right and left inner shafts 66, 68 and into gear pin apertures 92 on the gear wheel 70 such that the gear wheel 70 is capable of relative movement with respect to the right and left inner shafts 66, 68. The angled portions 78 and 86 of the right and left inner shafts 66, 68 are aligned as are the elongated notches 76 and 84.

The assembly of the inner shaft assembly 44 to the jaw assembly 12 will now be described in reference to FIG. 4. The distal end of the right inner shaft 66 is connected to the right jaw piece 18 by disposing the right jaw piece 18 into the pronged distal end 72 of the right inner shaft 66 and passing a shaft pin into the shaft pin apertures 74 of the right inner shaft 66 and into the shaft pin aperture 26 of the right jaw piece 18 such that the right inner shaft 66 is capable of relative movement with respect to the right jaw piece 18. The distal end of the left inner shaft 68 is connected to the left jaw piece 20 by disposing the pronged distal end 80 into the left jaw piece 20 (in particular, disposing the pronged distal end 80 into the right jaw receiving portion 32 of the left jaw piece 20) and passing a shaft pin into the shaft pin apertures 82 of the left inner shaft 68 and into the shaft pin apertures 36 of the left jaw piece 20 such that the left inner shaft 68 is capable of relative movement with respect to left jaw piece 20.

The assembly of the inner shaft assembly 44 to the outer shaft 42 will, now be discussed. The inner shaft assembly 44 is inserted into the inner shaft assembly receiving portion 48 of the outer shaft 42. A first jaw pin is passed into the upper jaw pin aperture 56 of the outer shaft 42 and into the upper jaw pin aperture 38 of the left jaw piece 20 and a second jaw pin is passed into the lower jaw pin aperture 56 of the outer shaft 42 and into the lower jaw pin aperture 38 of the left jaw piece 20 securing the left jaw piece 20 to the outer shaft 42 such that relative movement of the left jaw piece 20 and outer shaft 42 is permitted. Fasteners are passed through the connecting apertures 54 of the outer shaft 42 and into the apertures created by the conjunction of notches 76 and 84 of the adjacent right and left inner shafts 66, 68 to connect the inner shaft assembly 44 to the outer shaft 42 such that the right and left inner shafts 66, 68 are capable of relative movement with respect to the outer shaft 42.

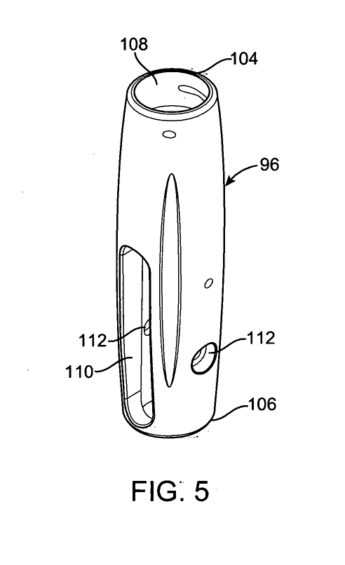

The handle assembly 16 will now be described in reference to FIGS. 5-8. The handle assembly 16 includes a handle 96, a trigger 98, a knob 100, spring 102 and a number of fasteners for connecting the elements together.

With reference first to FIG. 5, the handle 96 of the handle assembly 16 will now be described. The handle 96 has a proximal end 104 and a distal end 106. The handle 96 includes a bore 108 opening at and extending between the proximal end 104 and the distal end 106. A pair of oppositely disposed side windows 110 and at least one aperture 112 are also formed in the handle 96. A pair of oppositely disposed apertures 112 is employed for receiving fasteners to secure the gear wheel 70 to the handle 96.

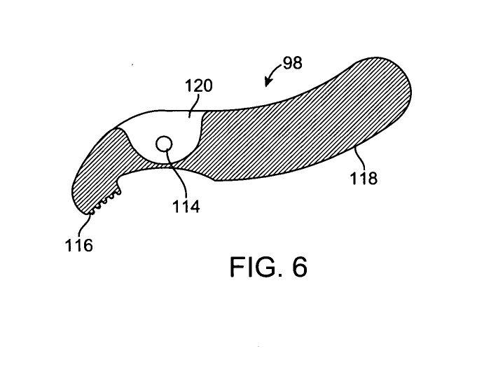

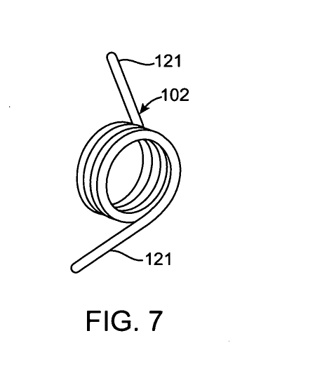

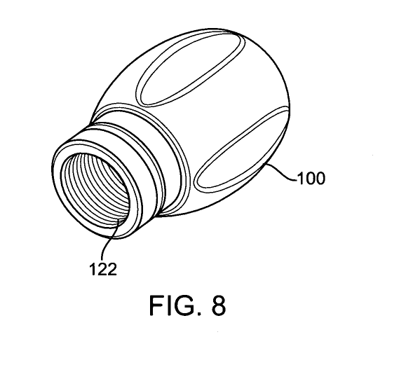

Referring now to FIG. 6, the trigger 98 of the handle assembly 16 will now be described. The trigger 98 includes an aperture 114, an engaging portion 116, a lever portion 118 and a spring receiving portion 120. The aperture 114 forms a pivot point for the trigger 98 once connected via a pin to the handle 96. The engaging portion 116 includes a toothed surface for contacting the toothed gear wheel 70 in a complementary fashion such that the toothed engaging portion 116 interlocks with the toothed periphery 88 of the gear wheel 70 for arresting rotation of the gear wheel 70. The lever portion 118 is configured with respect to the handle 96 for conveniently releasing the trigger 98. The spring receiving portion 120 is configured to receive a spring 102 of the like shown in FIG. 7.

Referring now to FIG. 7, there is shown a spring 102 for biasing the trigger 98 with respect to the handle 96. The spring 102 includes two legs 121 configured to be disposed inside the handle 96 and inside the spring receiving portion 120 such that actuation of the trigger 98 results in an opposed spring force biasing the trigger 98 towards the gear wheel 70.

Turning now to FIG. 8, there is shown a knob 100 of the handle assembly 16. The knob 100 includes a threaded bore 122 configured to receive the threaded portion 52 of the outer shaft 42.

The assembly of the handle assembly 16 and of the shaft assembly 14 to the handle assembly 16 will now be discussed. The proximal ends of the right and left inner shafts 66, 68 of the inner shaft assembly 44 are inserted at the distal end 106 into the bore 108 of the handle 96. The gear wheel 70 is inserted into the side window 110 of the handle 96 and the proximal ends of the right and left inner shafts 66, 68 are connected to the wheel 70 by inserting the right and left inner shafts 66, 68 into the inner shaft assembly receiving portion 94 of the gear wheel 70 and passing gear pins (not shown) into gear pin apertures of the right and left inner shafts 66, 68 and into gear pin apertures 92 on the gear wheel 70 such that the gear wheel 70 is capable of relative movement with respect to the right and left inner shafts 66, 68 within the constraints of the wedged-shaped inner shaft receiving portion 94. The gear wheel 70 is connected to the handle 96 by passing a fastener into the gear wheel apertures 112 in the handle 96 and the central bore 90 of the gear wheel 70. The trigger 98 and spring 102 are disposed inside the handle 96 and gear wheel 70 such that the spring 102 exerts a spring force against the trigger 98 forcing the toothed engaging portion 116 of the trigger 98 to contact the toothed gear wheel 70. A pin secures the trigger 98 to the handle 96 such that the trigger 98 is allowed to pivot about the pin with respect to the handle 96. The proximal end 58 of the outer shaft 42 extends from the bore 108 and beyond the proximal end 104 of the handle 96. The knob 100 is threadingly connected to the threaded portion 52 of the outer shaft 42. The distal end of the knob 100 is disposed inside the bore 108 of the handle 96 and secured thereto via pins passing through the handle and into a recess formed around the knob 100. In one variation, an impaction knob 124 (shown in FIG. 1d) is attached to the proximal end 58 of the outer shaft 42 and serves as an impaction surface for hammering the inserter 10 into position.

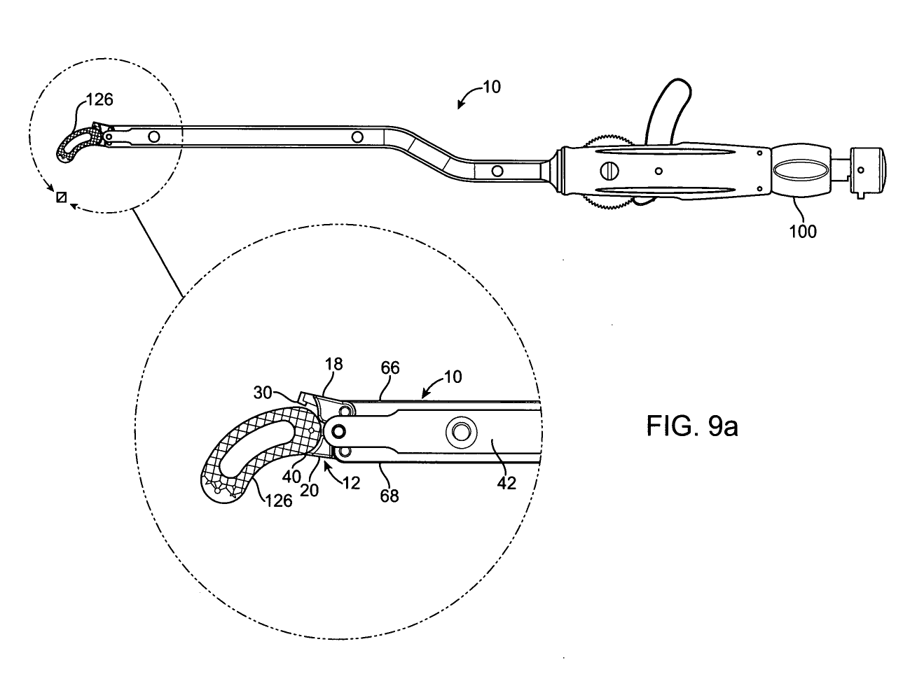

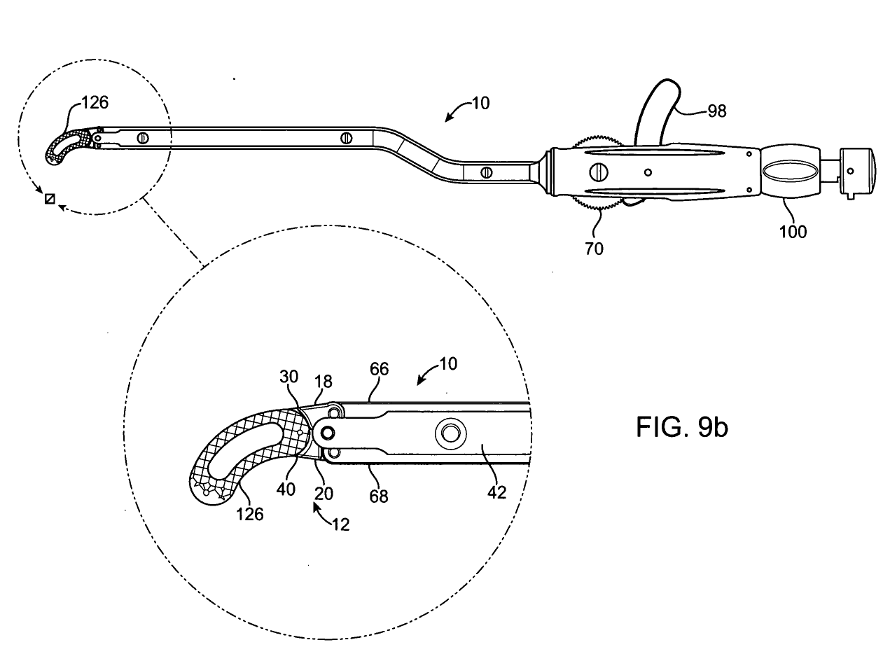

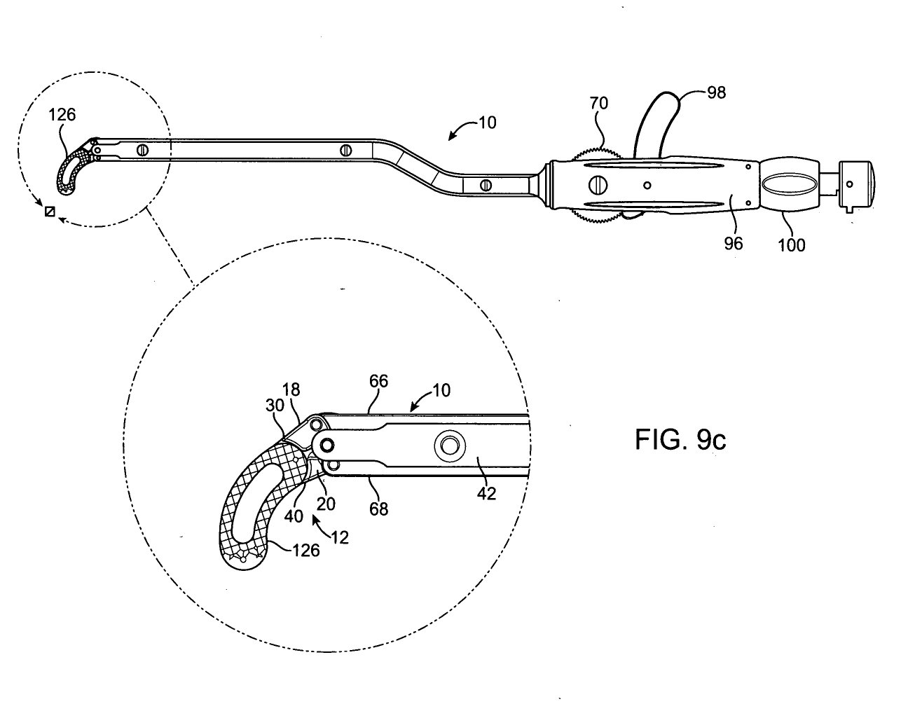

Operation of the inserter instrument 10 will now be discussed with initial reference to FIGS. 9a, 9b and 9c. Referring firstly to FIG. 9a, an interbody spacer 126 having engaging apertures (not shown) is shown in juxtaposition with the distal end of the inserter 10 with the jaw assembly 12 in an open position in which the jaws 18, 20 are spread apart and unlocked. The typical spacer 126 includes a body formed by a wall extending about a central cavity. The cavity extends between and opens at an upper bearing surface and a lower bearing surface. The upper and lower bearing surfaces contact the adjacent vertebral endplates to support the adjacent vertebrae when the spacer is implanted into the spinal disc space. The bearing surfaces include grooves or texturing formed to facilitate engagement with the vertebral endplates and resist the spacer from migrating within the disc space. The implant includes a convexly curved anterior wall and an opposite concavely curved posterior wall. These wall portions are connected by a convexly curved leading end wall and a convexly curved trailing end wall. The overall shape provides a banana or kidney type shape for the spacer.

The spacer 126 includes spacer engaging apertures (not shown) configured to be aligned with the spacer engaging features 30, 40 of the jaw assembly 12. The knob 100 of the handle assembly 16 is rotated such that the threaded engagement with the outer shaft 42 draws the outer shaft 42 into the bore 108 of the handle 96 and moves the jaw pin 22 along with it, thereby angulating the jaws 18, 20 towards each other into a closed position. In the closed or locked position, the spacer engaging features 30, 40 are securely clamped to the spacer 126 as shown in FIG. 9b. The spacer 126 is unclamped from the inserter 10 by rotating the knob 100 in the opposite direction.

Releasing the trigger 98 unlocks the gear wheel 70 which is then rotated with respect to the handle 96 by the user to articulate the jaws 18, 20. Rotation of the gear wheel 70 in one direction moves one of the right or left inner shafts 66, 68 proximally and moves the other of the right or left inner shafts 66, 68 distally, thereby, angulating the jaw assembly 12 as shown in FIG. 9c. FIG. 9c depicts the right inner shaft 66 moved distally and the left inner shaft 68 moved proximally and, as a result, the jaw assembly 12 angled with respect to the outer shaft 42 towards the left inner shaft 68 with relative rotation of the gear wheel 70. The gear wheel 70 rotation is permitted while the spring-biased trigger 98 is released from being locked onto the gear wheel 70. The gear wheel 70 is rotatable in an opposite direction, while the trigger 98 is unlocked from the gear wheel 70, to bring the jaw assembly 12 into a normal position or angulated beyond the normal and towards the right inner shaft 66 opposite of that shown in FIG. 9c. Any number of angular positions within the range permitted by the wedge-shaped inner shaft assembly receiving portion 94 of the gear wheel 70. The jaw assembly 12 can be locked at any point along its range of angulation by releasing the trigger 98 such that it engages the gear wheel 70 to lock it in place. Hence, the surgeon is able to angulate the spacer 126 relative to the instrument from, for example, a first orientation such as that shown in FIG. 9b to a second orientation such as that shown in FIG. 9c or any other position within its span of angulation. With the trigger 98 engaged by the user to thereby release the trigger 98 and free rotation of the gear wheel 70, angulation is achieved by the user turning the gear wheel 70 directly. Alternatively, angulation is achieved by the user taking advantage of the friction force on the implant 126 when placed between two vertebral bodies to hold the implant 126 in place while the entire instrument 10 is angulated with respect to the substantially stationary implant 126. For example, a spinal implant 126 is first connected to the inserter 10 as described above. The inserter 10 and attached implant 126 is then inserted into a patient through an incision and placed in between tissue or bony anatomy. If the implant is destined for placement between vertebral bodies, a measurement is typically made beforehand to determine the size of the implant most suitable for the particular patient anatomy. With the appropriately sized implant attached to the inserter and inserted in the target disc space, the vertebral bodies adjacent to the disc space will exert some force on the implant and hold it substantially in place, that is, there will be some resistance on the implant when inserted between the vertebral bodies or other areas of tight tissue. This resistance is employed by the surgeon user to hold the implant in place. With the implant held in place by friction from the patient anatomy, the gear wheel 70 is freed to rotate by moving the trigger and then angulating the entire inserter 10 relative to the implant 126 to achieve angulation. The desired angulation can then be locked and the surgeon user may then re-position the spacer 126 along another axis of insertion. Another axis or angle of insertion may be desirable for generating more insertive force, navigating the implant into position or for simply pinpointing placement of the spacer in the perfect location. Such angulation makes the instrument 10 useful for easing and facilitating insertion, positioning and implantation of the spacer 126 inside the patient. Advantageously, the trigger 98 may be repeatedly disengaged and re-engaged and the gear wheel rotated in one direction or another direction relative to the handle 96 as needed by the surgeon in situ, in either or both of the two methods described above to conveniently orientate the spacer without losing hold of the intervertebral spacer. The spacer 126 can be released from the inserter 10 at any angle by rotating the knob 100 and the instrument removed from the patient.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

We claim:

1. An inserter for implanting a spinal implant, comprising:
a jaw assembly configured to attach to the spinal implant;
a shaft assembly connected to the jaw assembly; the shaft assembly including a shaft connected to a gear wheel; and
a handle assembly connected to the gear wheel and the shaft assembly such that the handle assembly is operable to open and close the jaw assembly to thereby connect to and release the spinal implant and operable to lock and unlock rotation of the gear wheel to permit angulation of the jaw assembly relative to the shaft assembly.

2. The inserter of claim 1 wherein the gear wheel includes a wedge-shaped shaft receiving portion with the shaft disposed therein; the wedge-shaped shaft receiving portion limiting the degree of rotation of the gear wheel with respect to the shaft and thereby limiting the degree of angulation of an attached spinal implant with respect to the shaft assembly.

3. The inserter of claim 1 wherein the gear wheel is rotatable with respect to the handle assembly to angulate a spinal implant attached to the jaw assembly with respect to the shaft.

4. The inserter of claim 1 further including a trigger configured to engage the gear wheel to lock the angulation in place and configured to release the gear wheel to permit angulation.

5. The inserter of claim 4 wherein the trigger is configured such that it is movable distally by the user to release the gear wheel to permit angulation.

6. The inserter of claim 1 further including a slap hammer surface at the proximal end.

7. The inserter of claim 1 wherein the shaft includes a right shaft and left shaft; the proximal end of the right shaft being connected to the gear wheel at a distance offset from the center of the gear wheel; the proximal end of the left shaft being connected to the gear wheel at a distance offset from the center of the gear wheel.

8. The inserter of claim 1 wherein the shaft assembly includes an angled portion such that the distal working end of the inserter is displaced from the proximal handle end by a distance such that the distal working end of the inserter is substantially unobstructed by the inserter's proximal end.

9. An inserter for implanting a spinal implant, comprising:
a jaw assembly configured to attach to a spinal implant;
a shaft assembly connected to the jaw assembly; the shaft assembly including:
an inner shaft comprising a right inner shaft and a left inner shaft;
an outer shaft; and
a gear wheel;
wherein the inner shaft has a distal end configured to connect with the jaw assembly; the outer shaft has a distal end configured to connect with the jaw assembly; the inner shaft is located in the outer shaft such that the inner shaft is movable with respect to the outer shaft; the proximal end of the right inner shaft is connected to the gear wheel; the proximal end of the left inner shaft is connected to the gear wheel;
a handle assembly comprising:
a first portion; the first portion being connected to the gear wheel such that the gear wheel is rotatable with respect to the first portion; and
a second portion connected to the first portion; the second portion being connected to the proximal end of the outer shaft such that the outer shaft is movable with respect to the second portion;
a trigger connected to the first portion such that it is movable with respect to the first portion to unlock rotation of the gear wheel to permit angulation of the jaw assembly and lock rotation of the gear wheel to arrest angulation of the jaw assembly.

10. The inserter of claim 9 wherein the jaw assembly includes a right jaw piece movably connected to the distal end of the right inner shaft and a left jaw piece movably connected to the distal end of the left inner shaft; the right and left jaw pieces being configured to attach to a spinal implant.

11. The inserter of claim 10 wherein the right jaw piece is movably connected to the left inner shaft.

12. The inserter of claim 11 wherein the left jaw piece is connected to outer shaft.

13. The inserter of claim 12 wherein the second portion of the handle assembly is operable to move the outer shaft proximally and distally with respect to the inner shaft to open and close the jaw assembly.

14. The inserter of claim 9 wherein, with the trigger unlocked from the gear wheel, the angle of the shaft assembly relative to the jaw assembly is changeable.

15. The inserter of claim 9 wherein the gear wheel includes a wedge-shaped inner shaft receiving portion that limits the rotation of the gear wheel with respect to the inner shaft assembly and the angulation of the jaw assembly relative to the shaft assembly.

16. The inserter of claim 9 wherein the second portion of the handle assembly is threaded to the proximal end of the outer shaft and rotation of the second portion translates the outer shaft proximally or distally to lock or unlock the jaw assembly from the spinal implant.

17. The inserter of claim 9 wherein the trigger includes a gear wheel engaging portion and the trigger is spring-biased into locking engagement with the gear wheel to arrest movement of the gear wheel.

18. A method for implanting a spinal implant comprising:
providing an instrument comprising:
a jaw configured to attach to a spinal implant;
a shaft connected to the jaw; and
a handle connected to the shaft;
wherein the jaw is operable via the handle to open and close to connect to the spinal implant and also operable via the handle to angulate an attached spinal implant with respect to the shaft and further operable to lock and unlock such angulation;
providing a spinal implant configured to attach to the instrument and sized for frictional engagement between spinal anatomy;
opening the jaw of the instrument;
placing the spinal impant in juxtaposition to the jaw;
closing the jaw;
connecting the instrument to the spinal implant;
creating an incision in a patient targeting spinal anatomy;
inserting the instrument with the attached spinal implant into the incision;
positioning the spinal implant between spinal anatomy such that the spinal anatomy exerts a frictional force on the spinal implant holding it substantially in place;
unlocking the instrument such that angulation of the implant relative to the shaft is permitted;
angulating the attached implant relative to the shaft while the implant is connected to the instrument;
locking the instrument such that angulation of the implant relative to the shaft is arrested;
re-positioning the spinal implant between the spinal anatomy;
opening the jaw;
removing the instrument from the incision; and
leaving the spinal implant located between the spinal anatomy.
